(19) United States
(12) Patent Application Publication
(10) Pub. No.: US 2009/0123581 A1
(43) Pub. Date: May 14, 2009

(54) COLLAGEN CROSS-LINKING AGENTS ON DENTAL RESTORATIVE TREATMENT AND PREVENTIVE DENTISTRY

(75) Inventor: Ana Karina Bedran-Russo, Chicago, IL (US)

Correspondence Address: AKERMAN SENTERFITT, P.O. BOX 3188, WEST PALM BEACH, FL, 33402-3188, US

(73) Assignee: THE BOARD OF TRUSTEES OF THE UNIVERSITY OF ILLINOIS, Urbana, IL (US)

(21) Appl. No.: 12/300,559

(22) PCT Filed: Jun. 8, 2007
(86) PCT No.: PCT/US2007/070809

§ 371 Date: Nov. 12, 2008

Related U.S. Application Data
(60) Provisional application No. 60/812,664, filed on Jun. 9, 2006.
(60) Provisional application No. 60/918,640, filed on Mar. 16, 2007.

(51) Int. Cl. A61K 36/87; A61K 38/00; A61P 41/00

(52) U.S. Cl. 424/766; 514/2

(57) ABSTRACT

The invention relates to the development of compositions and methods for increasing the amount of collagen cross-linking in a mammalian tissue. A typical composition as described herein includes at least one cross-linking agent such as a bioflavonoid compound (e.g., proanthocyanidin), a grape seed extract, a Casein Phosphopeptide-amorphous Calcium Phosphate, or an iridoid compound (e.g., genipin) in an amount effective for increasing collagen cross-linking in the mammalian tissue in a pharmaceutically acceptable carrier. A typical method for increasing the amount of collagen cross-linking in dentin in a mammalian tooth includes the steps of preparing the surface of the tooth to be treated; and applying a composition including at least one of: a bioflavonoid compound, a grape seed extract, a Casein Phosphopeptide-amorphous Calcium Phosphate, and an iridoid compound in a pharmaceutically acceptable carrier to the tooth surface for a time period of 0.0001 hours to about 4 hours. In some embodiments, two or more cross-linking agents are included in the compositions described herein. The compositions and methods as described herein are particularly useful for applying to dentin in a mammalian tooth requiring a restorative procedure for improving the mechanical properties of restoration interfaces to withstand degradation over time. Compositions containing one of the collagen cross-linking agents as described herein were applied to dentin collagen and resulted in a significant improvement in ultimate tensile strength indicating the value of these compositions in restorative dentistry. The compositions and methods described herein will also find use in preventive dentistry applications, and can be applied to sound dentin, caries-affected dentin, and dentin that is impaired, weak, or degraded in any way.

### CROSS REFERENCE TO RELATED APPLICATIONS

The present application claims the priority of U.S. provisional patent application No. 60/812,664 filed Jun. 9, 2006, and U.S. provisional patent application No. 60/918,640 filed Mar. 16, 2007, and PCT Application No. PCT/US07/070,809 filed Jun. 8, 2007.

### STATEMENT AS TO FEDERALLY SPONSORED RESEARCH

The invention was funded by National Institutes of Health (NIH) grant NIH NIDCR DEO17740-01. The government has an interest in the invention.

### FIELD OF THE INVENTION

The invention relates generally to the field of dentistry. More particularly, the invention relates to compositions and methods for increasing the amount of collagen cross-linking in a mammalian (e.g., human) tissue.

### BACKGROUND

Adhesive restorations are routinely used to replace carious dental tissue, missing enamel and/or dentin in cervical areas, fractured tooth, and replacement of defective restorations. Adhesive restoratives consist of a combination of a restorative material such as composite resin and an adhesive system which will bond the restorative material to the tooth structure. Dentin is a complex mineralized tissue arranged in an intricate 3-dimensional frame composed of tubules extending from the pulp to the dentino-enamel junction, intratubular and peritubular dentin. It consists of a complex composition in which 70% (by weight) of its bulk is mineral, 20% is organic component and 10% is water. Of the organic matrix, fibrillar type I collagen accounts for 90% while the remaining 10% consists of non-collagenous proteins such as phosphoproteins and proteoglycans. Among all types of collagen, type I collagen is the predominant genetic product and it is an important molecule to provide tissues and organs with tensile strength, form and cohesiveness. In dentin, collagen is composed of inter- and intra-molecular cross-links that are important in providing tissue matrices with tensile strength, elasticity and resistance against enzymatic degradation. Several synthetic and natural chemicals increase the number of inter- and intra-molecular collagen cross-links. During restorative procedures, dentin is etched prior to or concomitant to the application of a primer/adhesive system, thus exposing collagen. In the fully-expanded state, the adhesive monomers then penetrate into the collagen network forming a hybrid layer in situ that is believed to be essential for dentin bonding. For effective bonding, therefore, the stability and maintenance of dentin collagen are important.

The successful approach for dentin adhesion relies on the application of phosphoric acid to demineralize dentin, rinsing with water, blot drying to maintain dentin moisture, application of a primer/adhesive agent, and finally placement of a composite resin. These adhesive systems have proved to be clinically successful; however, short-term failures have been reported due to breakdown in the tooth/restoration interface. Degradation of dentin/restoration bonded interfaces contributes to the decline in bond strengths produced by dentin adhesives over time leading to possible failure of the restoration. The degradation of the unprotected demineralized collagen matrix is partially associated with the failure of the bonding procedures. Failure in the tooth/restoration interface leads to formation of pathways in which oral fluid, bacterial products and endogenous proteolytic enzymes (e.g.: Matrix Metalloproteinases—MMPs) can infiltrate and degrade the interface components. This degradation can result in the development of secondary caries, sensitivity, and pulpal inflammation, leading to replacement of the restoration by removal of the old restoration plus substrate (usually decayed structures).

Even with all the advances in the development of restorative systems, a composite resin restoration lasts an average of 7 years in posterior teeth (Nakabayashi N. Int Dent J 35: 145-54, 1985; Han B, et al. J Biomed Mater Res 65A:118-124, 2003; Sung H-W, et al. J Biomed Mater Res 47:116-126, 1999; Sung H-W, et al. J Biomed Mater Res 64A:427-438, 2003; Ritter A V, et al. Eur J Oral Sci 109:348-53, 2001). Clinical studies have reported that approximately 50% of all restorations placed by dentists replace existing restorations (Nakabayashi N., Int Dent J 35:145-54, 1985; Han B, et al., J Biomed Mater Res 65A:118-124, 2003; Sung H-W, et al. J Biomed Mater Res 47:116-126, 1999; Sung H-W, et al., J Biomed Mater Res 64A:427-438, 2003; Ritter A V, et al. Eur J Oral Sci 109:348-53, 2001). Failure at the interface (secondary caries) is the primary reason given for replacement of composite restorations and accounts for 30-60% of all placed restorations (Han B, et al. J Biomed Mater Res 65A:118-124, 2003; Sung H-W, et al. J Biomed Mater Res 47:116-126, 1999; Ritter A V, et al. Eur J Oral Sci 109: 348-53, 2001).

Therefore, there is a need in the art to improve the restoration interface in order to minimize replacement of restorations and consequently improve health care and reduce health care costs.

### SUMMARY

The invention relates to the development of compositions and methods for increasing the amount of collagen cross-linking in a mammalian tissue (e.g., dentin, bone). A typical composition as described herein includes at least one cross-linking agent, such as a bioflavonoid compound (e.g., proanthocyanidin), a grape seed extract, a Casein Phosphopeptide-amorphous Calcium Phosphate (CPP-ACP), a synthetic collagen cross-linker or an iridoid compound (e.g., genipin) in an amount effective for increasing collagen cross-linking in the mammalian tissue in a pharmaceutically acceptable carrier. In some embodiments, two or more cross-linking agents are included. The compositions as described herein are particularly useful for applying to type I collagen in the dentin of a mammalian tooth requiring a restorative procedure (e.g., a bonding procedure) for improving the mechanical properties of restoration interfaces to withstand degradation over time. The integrity of fibrillar type I collagen is an important issue when bonding to dentin structure since it is the main dentin component involved during bonding procedures in restorative dental treatments. The contribution of exogenous collagen cross-links provided by compositions of the invention containing collagen cross-linking agents on the properties of dentin and consequently on the durability of adhesive restorative dental treatments was examined. Compositions containing one of the above-mentioned collagen cross-linking agents were applied to dentin collagen and resulted in a significant improvement in ultimate tensile strength (UTS) indicating the value of these compositions in restorative dentistry. The compositions and methods described herein will also find use in preventive dentistry applications, and can be applied to all tooth surfaces, including sound enamel, decalcified enamel, sound dentin, caries-affected dentin, and dentin that is impaired, weak, or degraded in any way.

Accordingly, the invention features a composition for increasing collagen cross-linking in a mammalian tissue including at least one of: a bioflavonoid compound, a grape seed extract, a Casein Phosphopeptide-amorphous Calcium Phosphate, a synthetic collagen cross-linker and an iridoid compound in a pharmaceutically acceptable carrier. The at least one of: a bioflavonoid compound, a grape seed extract, a Casein Phosphopeptide-amorphous Calcium Phosphate, a synthetic collagen cross-linker and an iridoid compound is present in an amount effective for increasing collagen cross-linking in the mammalian tissue. The at least one of: a bioflavonoid compound, a grape seed extract, a Casein Phosphopeptide-amorphous Calcium Phosphate, a synthetic collagen cross-linker and an iridoid compound can be a bioflavonoid compound such as a proanthocyanidin. The at least one of: a bioflavonoid compound, a grape seed extract, a Casein Phosphopeptide-amorphous Calcium Phosphate, a synthetic collagen cross-linker and an iridoid compound can be a synthetic collagen cross-linker such as glutaraldehyde. The composition can include a grape seed extract and a Casein Phosphopeptide-amorphous Calcium Phosphate. The at least one of: a bioflavonoid compound, a grape seed extract, a Casein Phosphopeptide-amorphous Calcium Phosphate, a synthetic collagen cross-linker and an iridoid compound can be any of a number of iridoid compounds, including: genipin, canthoside, caudatoside, saprosmoside, bis-iridoid, aucubin, gardenoside, alpha-iridodiol, geniposide, villoside, patrinoside aglycone, 11-deoxy patrinoside aglycone, gardenogenins, deacetylasperulosidic acid methylester genins, scandosife methylester genin, rehmannoside, harpagoside, harpagide, loganin, loganic acid, cantleyoside, secologanin, and secologanin-dimethylacetal. The at least one of: a bioflavonoid compound, a grape seed extract, a Casein Phosphopeptide-amorphous Calcium Phosphate, a synthetic collagen cross-linker and an iridoid compound can be a grape seed extract which includes proanthocyanidins.

The mammalian tissue can be dentin, and the collagen can be type I collagen. A pharmaceutically acceptable carrier can be distilled water, phosphate buffered solution, a solvent (e.g., alcohol), a gel (e.g., carbopol), and an acid (e.g., phosphoric acid, and citric acid). The at least one of: a bioflavonoid compound, a grape seed extract, a Casein Phosphopeptide-amorphous Calcium Phosphate, a synthetic collagen cross-linker and an iridoid compound can be a grape seed extract and the amount effective for increasing collagen cross-linking in the mammalian tissue can be about 0.001% to about 30% weight by volume. The at least one of: a bioflavonoid compound, a grape seed extract, a Casein Phosphopeptide-amorphous Calcium Phosphate, a synthetic collagen cross-linker and an iridoid compound can be an iridoid compound such as genipin and the amount effective for increasing collagen cross-linking in the mammalian tissue can be about 0.001% to about 5% weight by volume. The at least one of: a bioflavonoid compound, a grape seed extract, a Casein Phosphopeptide-amorphous Calcium Phosphate, a synthetic collagen cross-linker and an iridoid compound can be a synthetic cross-linker such as glutaraldehyde and the amount effective for increasing collagen cross-linking in the mammalian tissue can be about 0.001% to about 25% weight by volume.

In another aspect, the invention features a method of increasing collagen cross-linking in dentin in a mammalian tooth. The method includes the steps of: (a) preparing the surface of the tooth to be treated; and (b) applying a composition including at least one of: a bioflavonoid compound, a grape seed extract, a Casein Phosphopeptide-amorphous Calcium Phosphate, and an iridoid compound in a pharmaceutically acceptable carrier to the tooth surface for a time period of 0.0001 hours to about 4 hours. The at least one of: a bioflavonoid compound, a grape seed extract, a Casein Phosphopeptide-amorphous Calcium Phosphate, a synthetic collagen cross-linker and an iridoid compound is present in an amount effective for increasing collagen cross-linking in the mammalian tissue. The step (a) of preparing the tooth surface can include etching the tooth surface with a reagent (e.g., phosphoric acid). The at least one of: a bioflavonoid compound, a grape seed extract, a Casein Phosphopeptide-amorphous Calcium Phosphate, a synthetic collagen cross-linker and an iridoid compound can be a grape seed extract. The at least one of: a bioflavonoid compound, a grape seed extract, a Casein Phosphopeptide-amorphous Calcium Phosphate, a synthetic collagen cross-linker and an iridoid compound can be an iridoid compound (e.g., genipin).

Also featured within the invention is a method of performing a restorative procedure on a mammalian tooth. The method includes the steps of: (a) preparing the surface of the tooth to be treated; (b) applying a composition including at least one of: a bioflavonoid compound, a grape seed extract, a Casein Phosphopeptide-amorphous Calcium Phosphate, and an iridoid compound in a pharmaceutically acceptable carrier to the tooth surface for a time period of 0.0001 hours to about 4 hours, wherein the at least one of: a bioflavonoid compound, a grape seed extract, a Casein Phosphopeptide-amorphous Calcium Phosphate, a synthetic collagen cross-linker and an iridoid compound is present in an amount effective for increasing collagen cross-linking in the tooth; and (c) bonding a restorative material to the treated tooth surface.

Unless otherwise defined, all technical terms used herein have the same meaning as commonly understood by one of ordinary skill in the art to which this invention belongs.

By the term “iridoid compound” is meant a compound recognized as iridoid as a chemical compound or an equivalent of iridoid as a chemical compound by a person of ordinary skill in the art. The term “iridoid compound” is intended to cover all iridoid glucosides and aglycones. Examples of iridoid compounds include aucubin, gardenoside, alpha-iridodiol, geniposide, genipin, villoside, patrinoside aglycone, 11-deoxy patrinoside aglycone, gardenogenins, deacetylasperulosidic acid methylester genins, scandosife methylester genin, rehmannoside, harpagoside, harpagide, loganin, loganic acid, cantleyoside, secologanin, and secologanin-dimethylacetal. The iridoid compound can be derived from natural sources or synthetically made.

By the term “proanthocyanidin” is meant any oligomeric polyphenolic compound composed of bioflavonoids that have the ability to interact with proteins such as collagen. The term encompasses proanthocyanidins that are naturally occurring and synthetic. Naturally occurring proanthocyanidin is an extract from grape seeds. As used herein, the phrase “grape seed extract” means an extract of grape seeds, the extract containing proanthocyanidins. The terms “grape seed extract” and “proanthocyanidin” are used interchangeably herein.

As used herein, the term “genipin” is meant a compound recognized as genipin as a chemical compound or an equivalent of genipin as a chemical compound by a person of ordinary skill in the art. The term “genipin” is intended to cover derivatives, analog, stereoisomers and mixtures thereof. The genipin compound can be derived from natural sources or synthetically made.

By the term “bioflavonoid” is meant a flavonoid having biological activity in mammals.

As used herein, the terms “cross-linking agent” and “cross-linker” are intended to cover a chemical agent that could react with proteins, such as collagen, through covalent and hydrogen bonds.

Although compositions, methods and materials similar or equivalent to those described herein can be used in the practice or testing of the present invention, suitable compositions, methods and materials are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIGS. 1A-1C are a schematic illustration showing specimen preparation, treatment and testing. FIG. 1A illustrates specimen preparation. FIG. 1B illustrates specimen treatment. FIG. 1C illustrates specimen testing.

FIGS. 2A and 2B are a pair of graphs showing UTS values for demineralized dentin after 4 hours (2A) and 40 hours (1B) exposure. Same letters indicate no significant differences between groups (P<0.05) within each Figure.

FIGS. 3A and 3B are a pair of graphs showing UTS values for undemineralized dentin after 4 hours (3A) and 40 hours (4B) exposure. Same letters indicate no significant differences between groups (P<0.05) within each Figure.

FIG. 4A is a graph showing bond strength values of Single Bond placed in caries-affected dentin. Different letters indicate significant differences between groups (P<0.05).

FIG. 4B is a graph showing bond strength values of Single Bond placed in sound dentin. Different letters indicate significant differences between groups (P<0.05).

FIG. 5 is a diagram illustrating specimen selection, preparation, treatment, and testing.

FIG. 6 is a diagram illustrating specimen preparation and treatment.

FIG. 7 is a graph showing UTS values (MPa) for the groups evaluated. GD=glutaraldehyde, PA=proanthocyanidin.

FIG. 8 is a graph showing UTS values for control, glutaraldehyde-containing, and grape seed extract-containing samples.

### DETAILED DESCRIPTION

The invention encompasses compositions and methods relating to increasing the amount of collagen cross-linking in a mammalian tissue (e.g., dentin, bone). The compositions and methods described herein may be particularly useful for applying to the collagen in the dentin of a mammalian tooth requiring a restorative procedure such as use of fillings/material that adhesively bond to the tooth structures for improving the mechanical properties of and decreasing the enzymatic degradation of restoration interfaces. Due to their ability to decrease enzymatic degradation of collagen, these compositions and methods are also useful in preventive dentistry applications. During a disease process such as decay/caries formation, the bacteria in the oral environment release enzymes that degrade the collagen. Chemically modifying the tooth surface with collagen cross-linkers can decrease the susceptibility of dentin collagen degradation and consequently tooth degradation. The below described preferred embodiments illustrate adaptations of these compositions and methods. Nonetheless, from the description of these embodiments, other aspects of the invention can be made and/or practiced based on the description provided below.

Restorative Dentistry Methods

Methods involving conventional restorative dentistry techniques are described herein. Such techniques are generally known in the art and are described in detail in methodology treatises such as Adhesive Technology for Restorative Dentistry by Jean-Francoise Roulet, 2004, Quintessence Publishing, Chicago, Ill. and Protocols for Predictable Aesthetic Dental Restoration by Irfan Ahmad, 2006, 1^{st} edition, Blackwell Publishing Limited, Malden, Mass. Methods of applying cross-linking agents to dentin collagen are described, for example, in Bedran-Russo et al., J Biomed Mater Res B: Appl Biomater 80:268-272, 2007. Pharmaceutically acceptable carriers are well known in the art, and are described in “Remington's Pharmaceutical Sciences” by Alfonso R Gennaro, 18^{th} edition, 1995, Mack Publishing Company, Easton, Pa.

Compositions

The invention includes compositions for increasing the amount of collagen cross-linking in a mammalian tissue (e.g., dentin, bone). Compositions as described herein include at least one of: a bioflavonoid compound, a grape seed extract, CPP-ACP, a synthetic collagen cross-linker and an iridoid compound in a pharmaceutically acceptable carrier. The at least one of: a bioflavonoid compound, a grape seed extract, a synthetic collagen cross-linker and an iridoid compound is present in an amount effective for increasing collagen cross-linking in the mammalian tissue. In a typical embodiment in which a composition is applied to dentin for preparing a tooth for the application of a restoration, the composition can increase the amount of type I collagen cross-linking in the dentin, and thereby improve the mechanical properties of the restoration interface and decrease enzymatic degradation of the restoration interface. Collagen in biological tissue is strengthened by the formation of native cross-links, which provides the fibrillar resistance against enzymatic degradation as well as greater tensile properties. Type I collagen is present in a tissue as fibrils that are stabilized by covalent intermolecular cross-linking. Collagen fibrils are strengthened by contacting them with compositions containing one or more cross-linking agents as described herein in order to increase the mechanical properties of the collagen fibrils and to decrease enzymatic degradation of a restoration interface. A cross-linking agent as described herein can induce additional intra- and intermolecular cross-links in collagen, and/or cross-links between two adjacent microfibrils (intermicrofibrillar cross-links).

Compositions for increasing the amount of type I collagen cross-linking in dentin for improving the mechanical properties of and decreasing the enzymatic degradation of restoration interfaces as described herein include at least one cross-linking agent. Studies have shown that increased collagen cross-links, provided by cross-linking agents, can increase collagen resistance to enzymatic degradation, increase temperature denaturation and improve mechanical properties (J Biomed Mater Res; Han, et al.; 2003; 65A:118-124) (J Biomed Mater Res; Sung, et al.; 2003; 64A:427-438). Several synthetic (e.g., glutaraldehyde, tannic acid, carbodiimides, formaldehyde, etc.) and naturally occurring (e.g., flavonoids, genipin, proanthocyanidin, grape seed extract, etc.) cross-linking agents can induce the formation of inter- and intra-molecular collagen cross-links. Inter-microfibrillar cross-links are thought to affect the mechanical properties of the tissue, while the stability of the collagen fibril is determined by inter- and intra-molecular cross-links (Sung et al., J Biomed Mater Res; Sung, et al.; 2003; 64A:427-438).

Any suitable cross-linking agent can be used in compositions and methods as described herein. Cross-linking agents that are particularly useful include bioflavonoids, grape seed extract, glutaraldehyde, CPP-ACP and iridoid compounds. Bioflavonoids are flavonoids with biological activity in mammals and have been shown to promote a strong collagen matrix. Among these, grape seed extract and proanthocyanidin (also called pycnogenol, leukocyanidin, and leucoanthocyanin) obtained from grape seed extract have been shown to be beneficial to connective tissue. Proanthocyanidin is a naturally occurring plant metabolite widely available in fruits, vegetables, nut seeds, flowers and barks (Altern Med Rev, Fine; 2000; 5(2):144-51) (Curr Pharm Biotechnol, Joshi, et al.; 2001; 2(2):187-200). Due to its interactions with collagen, proanthocyanidin has the ability to induce collagen cross-linking. Proanthocyanidins were found to increase collagen synthesis and accelerate the conversion of soluble collagen to insoluble collagen during development (J Biomed Mater Res; Han, et al.; 2003; 65A:118-124) (J Rheumatol, Rao; 1983; 12(1):39-42) (Ital J Biochem, Cetta; 1977; 26(4):317-27). Furthermore, proanthocyanidins are widely used as natural antioxidants, free-radical scavengers and have proven to be safe in different clinical applications and as dietary supplements (Toxicol Lett., Sabino; 1999; 108(1):27-35).

Grape seed extract, a proanthocyanidin-based compound is commercially available from MegaNatural-Polyphenolics Ind (Madera, Calif.). In a typical composition of the invention, an effective amount of grape seed extract (proanthocyanidin) for increasing collagen cross-linking is a concentration of about 0.001 to about 30% (e.g., 0.001, 0.005, 0.01, 0.1, 1.0, 5.0, 10.0, 15.0, 20.0, 25.0, 30.0, 35.0%, etc.) weight by volume. The amount of grape seed extract (proanthocyanidin) in the composition can depend on the pH and the solvent (pharmaceutically acceptable carrier) used. In addition to grape seed extract, other proanthocyanidin-containing extracts that are functionally equivalent to grape seed extract can be used. Examples of additional proanthocyanidin-containing extracts include, but are not limited to, cocoa extract and cinnamon extract.

An effective amount of glutaraldehyde in a composition for increasing collagen cross-linking in dentin in a mammalian tooth is 0.001-25% (e.g., 0.001, 0.005, 0.01, 0.1, 1.0, 5.0, 10.0, 15.0, 20.0, 25.0, 28.0%, etc.) weight by volume.

Any iridoid compound having a crosslinking ability may be used in the compositions and methods described herein. Illustrative examples are the aglycones of geniposide, gardenoside, geniposidic acid, etc. Genipin which is the aglycone of geniposide, isolated from the fruits of Gardenia jasminoides Ellis, is typically included. Genipin has been used as a cross-linking agent in a number of biological applications due to its low cytotoxicity. Genipin is commercially available from Wako Pure Chemical Ind (Osaka, Japan). Alternatively, genipin may be obtained from the parent compound geniposide, which may be isolated from natural sources as described elsewhere (see e.g., Fujikawa et al., J Biotechnol Lett, 9: 697-70, 1987; Tsai T H, et al. J Liq Chromatogr 17: 2199-205, 1994; Sung, et al., Biomaterials, 20(19):1759-72, 1999). Genipin may be prepared from geniposide by oxidation followed by reduction and hydrolysis or by enzymatic hydrolysis. Alternatively, racemic genipin may be prepared synthetically. Any stereoisomer or mixture of stereoisomers of genipin may be used as a crosslinking agent, in accordance with the present invention. Iridoid compounds may be prepared in accordance with the disclosures in Bedran-Russo et al., J Biomed Mater Res B Appl Biomater 80:268-272, 2007; and Sung et al., J Biomed Mater Res A, 64:427-438, 2003. An effective amount of iridoid compound for increasing collagen cross-linking is in the range from about 0.001 to about 5% (e.g., 0.001, 0.005, 0.01, 0.05, 0.1, 0.5, 1.0, 1.5, 2.0, 2.5, 3.0, 3.5, 4.0, 4.5, 5.0, 5.5%). Typically, the concentration (i.e., effective amount) is in the range from about 0.5% to about 1%.

In some embodiments, CPP-ACP can be included in a composition for increasing the amount of type I collagen cross-linking in dentin for improving the mechanical properties of and decreasing the enzymatic degradation of restoration interfaces. CPP-ACP, often referred to as CPP-ACP nanocomplexes, is derived from bovine milk protein, casein and calcium and phosphate and has been shown to be an effective remineralizing agent (Mazzaoui et al., J Dent Res 82(11):914-918, 2003; U.S. Pat. Nos. 6,846,500 and 6,733,818). An effective amount of CPP-ACP for increasing collagen cross-linking is in the range from about 0.01% to about 35% (e.g., 0.01, 0.05, 1.0, 5.0, 10.0, 15.0, 20.0, 25.0, 30.0, 35.0%) weight by volume.

A composition as described herein can have any suitable pH, but typically has a pH in the range of about 7.0 to about 7.5. A composition containing at least one collagen cross-linking as described herein that is to be added to a dental adhesive system, for example, can have a pH in the range of 1 to 8. In some embodiments, one or more anti-oxidants (e.g., vitamin C) can be included in a composition as described herein to extend the shelf life of the composition.

In some embodiments of a composition or method for increasing the amount of type I collagen cross-linking in dentin for improving the mechanical properties of and decreasing the enzymatic degradation of restoration interfaces, two or more cross-linking agents are included. For example, grape seed extract (proanthocyanidin) and glutaraldehyde can be included in a composition for increasing the amount of type I collagen cross-linking in dentin. In this example, grape seed extract (proanthocyanidin) is included at a concentration between about 0.001% and about 30.0% weight by volume, and glutaraldehyde is included at a concentration between about 0.5% and about 25% weight by volume. Such a composition can be prepared by any suitable method. For example, glutaraldehyde at a concentration of about 5% (weight by volume) can be diluted in water (e.g., distilled water), and grape seed extract at a concentration of about 6.5% (weight by volume) is added to the solution. The pH is then adjusted with a suitable reagent such as potassium hydroxide to a pH of about 7.0 to about 7.5. Additional collagen cross-linking combinations can also be used. For example, a composition including both genipin and glutaraldehyde can be used. As another example, a composition including both genipin and grape seed extract can be used.

Compositions for increasing collagen cross-linking in the dentin of a mammalian tooth can come in a number of forms. Compositions can come in the form of a toothpaste or a mouth wash. Additionally, compositions can come in the form of a gel, or a varnish that is applied to a subject's tooth by a dentist. Compositions can be applied to teeth by any suitable means, including for example, a plastic or rubber tray that is formed to fit over a subject's teeth and to hold a suitable amount of a composition.

Methods of Increasing the Amount of Type I Collagen Cross-Linking in Dentin

A typical method of increasing the amount of type I collagen cross-linking in dentin includes the steps of preparing the surface of the tooth to be treated, and applying a composition including at least one collagen cross-linking agent as described herein to the tooth surface. Typically, preparing the tooth surface includes etching the tooth surface with a reagent such as phosphoric acid. Application of the composition including at least one collagen cross-linking agent as described herein to the tooth surface typically involves applying a composition in the form of a solution or a gel. After the composition is applied, restorative procedures can be performed. For example, a tooth to which has been applied a composition including at least one collagen cross-linking agent as described herein can be rinsed, excess water can be removed, and an adhesive system can be applied. Generally, an adhesive system includes a primer and an adhesive, or a primer/adhesive reagent. In a restorative procedure, a composition including at least one collagen cross-linking agent as described herein can be used as a pre-treatment. For example, the composition can be applied as a cleanser before a resin cement or self-etching adhesive system is applied. In some embodiments, a composition including at least one collagen cross-linking agent as described herein is incorporated in one of the components of an adhesive system (e.g., to the phosphoric acid, primer or adhesive) before being applied to a tooth surface, and in these embodiments, no additional steps may be necessary. The time during which a composition including (e.g., to the phosphoric acid, primer or adhesive) can vary according to the concentration used and the application procedure.

In accordance with the methods described herein, the amount of the at least one cross-linking agent (e.g., iridoid compound, proanthocyanidin, etc.) used may be adjusted based on the use (i.e., restorative vs. preventive). In preventive applications, grape seed extract (proanthocyanidin) is typically included at a concentration of about 0.001% weight by volume to about 3% weight by volume, while genipin is typically included at a concentration of about 0.001% weight by volume to about 1% weight by volume.

In methods of increasing the amount of type I collagen cross-linking in dentin as described herein, any suitable dental adhesive can be used. Examples of suitable dental adhesives include Adper Single Bond Plus adhesive commercially available from 3M ESPE (St. Paul, Minn.) and One Step Plus dental adhesive commercially available from Bisco (Richmond, BC, Canada). Methods for preparing and applying dental adhesives are well known in the art, and are described, for example, in Duke E S, Compend Contin Educ Dent., 24(6):417-9, 421, 423-4:2003; Kugel G, Ferrari M., J Am Dent Assoc., 131 Suppl: 20S-25S:2000; and Breschi et al., Dent Mater., Apr. 16:2007 [Epub ahead of print].

Kits for Increasing the Amount of Type I Collagen Cross-Linking in Dentin

Also included within the invention is a kit for increasing the amount of Type I collagen cross-linking in dentin in a mammalian subject. Such a kit can be used in restorative procedures (e.g., stabilizing the restoration interface) and in preventive applications (e.g., preventing or slowing the decay of dentin). A typical kit for use in a restorative procedure contains a composition including grape seed extract in an amount effective to increase collagen cross-linking in a mammalian tissue, or a combination of grape seed extract and glutaraldehyde, each in an amount effective to increase collagen cross-linking in a mammalian tissue. In such a kit, the composition can be included in or combined with a dental adhesive system.

A typical kit for use in a preventive application contains a composition including grape seed extract in an amount effective to prevent dental decay or slow the progression of dental decay, or a combination of grape seed extract and fluoride, each in an amount effective to prevent dental decay or slow the progression of dental decay. In a kit for use in a preventive application, the composition is typically in the form of a toothpaste or mouth wash available over-the-counter, or in the form of a gel or varnish applied to a mammalian subject's tooth by a dentist. A kit as described herein can further include directions for using the composition contained within the kit.

### EXAMPLES

The present invention is further illustrated by the following specific examples. The examples are provided for illustration only and should not be construed as limiting the scope of the invention in any way.

### Example 1

### Application of Cross-Linkers to Dentin Collagen Enhances the Ultimate Tensile Strength

The stabilization of dentin collagen with biocompatible cross-linking agents are of clinical importance to improve dentin bond strength. The present study aimed to evaluate the effect of three collagen cross-linking agents on the ultimate tensile strength (UTS) of undemineralized and demineralized dentin.

Ten freshly extracted sound molars were sectioned into thick beams. The beams were either demineralized or kept undemineralized. Then, specimens were subdivided into four groups according to treatments: PBS solution—control; 5% glutaraldehyde (GD); 0.5% proanthocyanidin PBS solution (PA); and 0.625% genipin PBS solution (GE). To assess UTS, specimens were subjected to tensile forces at a crosshead speed of 1 mm/min. Statistical analysis was performed using two-way ANOVA and Fisher's PLSD test (p<0.05). Statistically significant increases in UTS were observed for demineralized dentin after PA and GE dentin treatment when compared to the control group. Dentin treated with GD showed no statistically significant differences in UTS when compared to the control. Mineralized dentin revealed no significant differences as compared to the control regardless of the collagen cross-linkers. The application of two naturally occurring cross-linkers, i.e. PA and GE, to dentin collagen significantly improved UTS indicating their potential value in restorative dentistry.

Materials and Methods

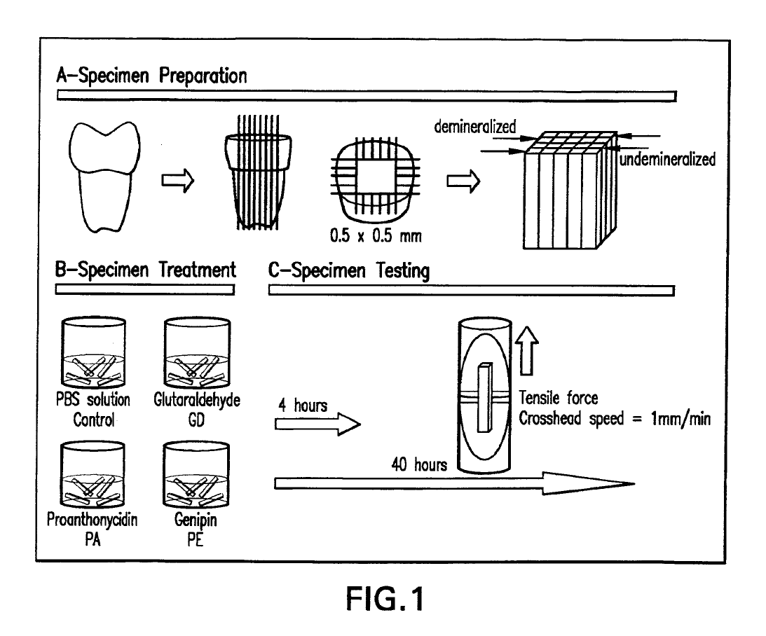

Fixation Processes: Ten sound freshly extracted human molars were used in this study. The teeth were thawed, cleaned of adhering soft tissues, and the occlusal surfaces were ground flat with #320 grit Silicon Carbide paper under running water to remove part of the enamel. Flattening of the occlusal surface by removal of the cusps enables accurate sectioning of samples in beam shape. Teeth were sectioned into 0.5±0.1 by 0.5±0.1 mm thick beams (n=20 beams per tooth) with a slow speed diamond wafering blade (Buehler-Series 15LC Diamond, Lake Bluff, Ill., USA) under constant water coolant (FIG. 1A).

The slabs were randomly divided into two groups: 1) undemineralized dentin and 2) demineralized dentin (FIG. 1A). In Group 2, dentin beams were immersed in 10% phosphoric acid solution (Fisher Scientific, New Jersey, N.J.) for a period of 5 hours. X-rays were taken from the specimens to verify complete tissue demineralization. Specimens were thoroughly rinsed with distilled water for 10 minutes to remove any residue of the phosphoric acid.

Specimens from groups 1 and 2 were subdivided according to the dentin treatment (FIG. 1B). In Treatment A, specimens were immersed in PBS solution (pH: 7.4) as a control. In Treatment B, specimens were immersed in 0.5% Proanthocyanidin (MegaNatural—Polyphenolics Ind.) PBS solution (PA) at a pH of 7.4. In Treatment C, specimens were immersed in 0.625% Genipin (Wako Pure Chemical Ind.) PBS solution (GE) at a pH of 7.4. In Treatment D, specimens were immersed in 5% glutaraldehyde solution (GD) (Fisher Scientific, New Jersey, N.J.) at a pH of 7.4. Specimens were kept in their respective solutions for 4 and 40 hours at room temperature. After the incubation periods, specimens were thoroughly rinsed with distilled and deionized water to remove any remains of cross-linking agents and buffer.

UTS testing: For UTS evaluation, the specimens were glued with a cyanoacrylate adhesive (Zapit—Dental Ventures of America Inc., Corona, Calif.) to a Ciucchi's jig, which was mounted on a universal testing machine (EZ Test, Shimazu Co., Kyoto, Japan) and subjected to microtensile testing at a crosshead speed of 1 mm/min (FIG. 1C). Means and standard deviations were calculated and expressed in microtensile bond strengths (MPa). Statistical analysis was performed using a two-way ANOVA and Fisher's PLSD tests (p<0.05).

TABLE 1
Ultimate tensile strength values in MPa [Mean (Standard deviation)] of
the demineralized dentin groups.

| | Dentin Treatment | | | | |
|---|---|---|---|---|---|
| | | Crosslinking agents | | | |
| Time | Control | PA | GE | GD | Pooled data |
| 4 hours | 10.8 | 17.7 | 11.0 | 13.1 | 13.2 A |
| | (4.9) | (9.4) | (3.8) | (6.6) | (6.2) |
| 40 hours | 10.6 | 21.9 | 18.2 | 9.1 | 15.0 A |
| | (4.7) | (7.0) | (6.2) | (2.4) | (5.1) |
| Pooled data | 10.7 C | 19.8 A | 14.6 B | 11.1 BC | |
| | (4.8) | (8.2) | (5.0) | (4.5) | |

Same letters equal no statistical significant difference (p > 0.05).
PA—proanthonycidin;
GE—genipin;
GD—glutaraldehyde

TABLE 2
Ultimate tensile strength values in MPa [Mean (Standard deviation)]
of the mineralized dentin groups.

| | Dentin Treatment | | | | |
|---|---|---|---|---|---|
| | | Crosslinking agents | | | |
| Time | Control | PA | GE | GD | Pooled data |
| 4 hours | 108.8 | 103.0 | 84.2 | 94.5 | 97.6 A |
| | (44.3) | (29.6) | (18.1) | (21.6) | (28.4) |
| 40 hours | 109.2 | 108.1 | 94.2 | 110.4 | 105.5 A |
| | (44.1) | (22.6) | (24.8) | (32.8) | (31.1) |
| Pooled data | 109.0A | 105.6 A | 89.2 A | 102.5 A | |
| | (44.2) | (26.1) | (21.4) | (27.2) | |

Same letters equal no statistical significant difference (p > 0.05).
PA—proanthonycidin;
GE—genipin;
GD—glutaraldehyde

Results:

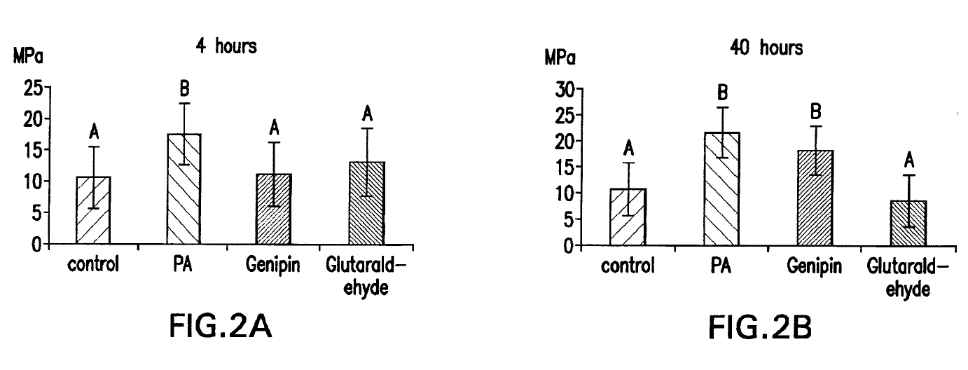

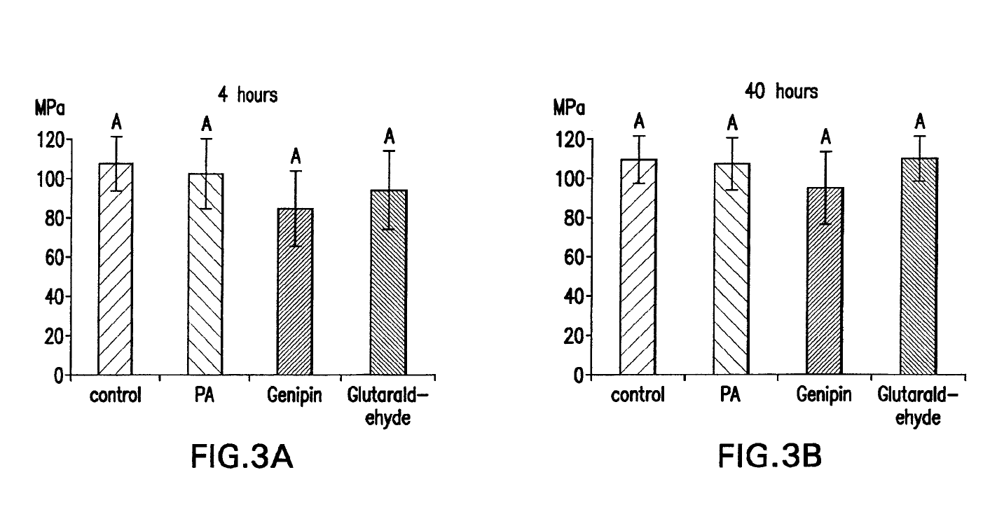

The mean UTS values and standard deviations (MPa±SD) for undemineralized and demineralized dentin are shown in Table 1 and Table 2, respectively and in FIG. 2 and FIG. 3, respectively. For demineralized dentin, a statistically significant (p<0.0001) interaction was observed between the factors studied (dentin treatment and time). The two periods of times used (4 and 40 hours) showed no statistically significant effect on the UTS values of demineralized dentin (p=0.2045) while the dentin treatment had a statistically significant effect on the results (p=0.0387). A highly significant increase in UTS values was observed after PA dentin treatment, compared to the control and the other two cross-linking agents. The increase of almost approximately 70% and 110% in the UTS values after PA treatment during 4 and 40 hours respectively, indicates a great potential of the agent to induce cross-links in the dentin collagen. Additional exposure to PA resulted in higher values, but they were not statistically different than the values obtained after 4 hours. GE treatment also resulted in a statistically significant increase in UTS values as compared to the control group, but was not significantly different from the GD treatment. Treatment with GE increased the UTS values by approximately 80% after 40 hours indicating a potential effect of this agent on stabilization of dentin collagen. The treatment of demineralized dentin with GD showed no statistically significant differences in UTS values when compared to the control group.

In mineralized dentin, no statistically significant (p=0.8988) interaction was observed between the factors studied. The times used (p=0.2999) as well as the dentin treatment (p=0.2707) did not significantly affect UTS bond strength values. There were no significant differences among the collagen cross-linkers as compared to the control when dentin was kept mineralized.

The results described herein demonstrated that naturally occurring cross-linking agents such as PA and GE are capable of stabilizing demineralized dentin collagen by inducing cross-links in dentin collagen and that PA and GE resulted in increased mechanical properties after tensile testing the demineralized matrix. Since an increase in the mechanical properties of collagen in the hybrid layer, an adhesive restorative interface, likely results in a prolonged life of restorations, the induction of cross-linking with those biocompatible compounds is useful in restorative dentistry as well as in preventive applications. An increase of the mechanical properties of interfaces is of major importance in restorative dentistry since long-term stability of restoration interfaces are still a challenge.

### Example 2

### Effect of Cross-Linking Agents on the Ultimate Tensile Strength of Dentin

Effect of an exogenous collagen cross-linking agent on dentin bonding: In order to analyze the contribution of exogenous collagen cross-linking to sound and caries-affected dentin bonding, demineralized dentin surfaces were treated with 5% glutaraldehyde and the restored specimens were subjected to microtensile bond strength testing.

Specimen preparation: Six human molars stored frozen (−20° C.) for less then one month were used. The teeth presented occlusal caries in which enamel was visually intact. The teeth were thawed, cleaned of adhering soft tissues, and the occlusal surfaces were ground flat with #320 and #600 grit Silicon Carbide (Sic) papers under running water to remove the enamel and expose flat middle dentin, respectively. To expose caries-affected dentin, a caries detector (Caries Detector—Kuraray Dental Co., Japan) was used for 10 seconds and thoroughly rinsed for 10 seconds. The surface was ground with #600 grit Sic until lightly pink dentin was exposed (caries-affected dentin). Then, the exposed dentin was etched with 37% phosphoric acid gel (3M ESPE, St. Paul, Minn.) for 15 seconds, thoroughly rinsed with water for 15 seconds, and kept moist. The dentin surface was then exposed to 5% glutaraldehyde solution for 5 minutes and thoroughly rinsed for 5 minutes to remove glutaraldehyde residues. The surface was blot dried using absorbent paper and an acetone-based dentin adhesive system (One Step—Bisco Dental Products, Schaumburg, Ill.) was applied, gently air dried for 10 seconds to remove the solvent and photo-polymerized for 10 seconds. A composite resin (Z250—3M ESPE) was used to build a crown incrementally in order to prepare the samples for testing. Control groups were treated in the same manner, except that 5% glutaraldehyde treatment was replaced by distilled water treatment.

Microtensile bond test (MTB): After 24 hrs in water at 37° C., the teeth were sectioned into 0.7±0.2 mm thick slabs, and the interfaces trimmed to an hourglass shape using a fine diamond bur to produce a cross-sectional area of 1 mm². The thickness of the slabs and the width of the trimmed surface were measured using a digital caliper (Fisher Scientific, New Jersey, N.J.) and specimens in sound and caries-affected dentin were obtained. All specimens were subjected to tensile forces at a crosshead speed of 1 mm/min using an EZ-Test testing machine (Shimadzu Co., Kyoto, Japan). MPa were determined by dividing the fracture load by the cross-sectional area of the interface.

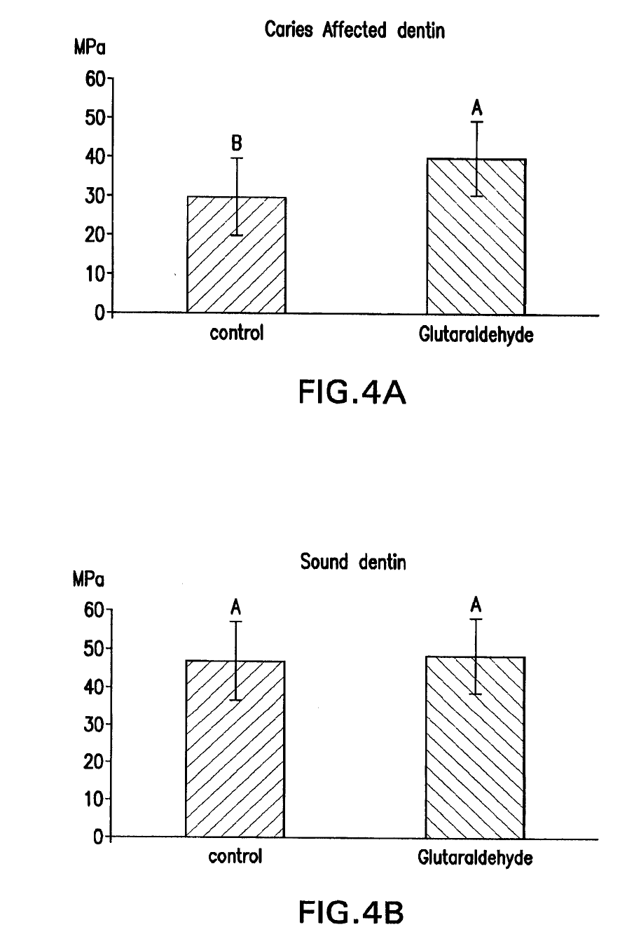

The results (FIGS. 4A and 4B) revealed a significant increase in bond strengths when the caries-affected demineralized dentin matrix was treated with 5% glutaraldehyde as compared to its respective control (p=0.0328). Glutaraldehyde treatment resulted in a 35% increase in the bond strength values. In contrast, no significant effect was observed when glutaraldehyde was applied to sound dentin (p=0.2395) (FIG. 4B). Glutaraldehyde is able to form collagen cross-links through the aldehyde functional groups that reacts primarily with the ε-amino groups of lysil or hydroxylysyl residues. When the adhesive system is placed in caries-affected dentin, a thicker hybrid layer is formed, thus not all collagens is encapsulated by the monomer, and abnormal tags are obliterated due to the presence of acid resistant mineral in the tubules. The increase in the mechanical properties of collagen after the use of cross-linking agents resulted in higher bond strength values of the restoration/caries-affected dentin interface. The lack of effect in the bond strength of sound dentin can be related to the thickness of the hybrid layer and the presence of collagen cross-linking precursors when compared to the caries-affected dentin tissue. These results indicate that the formation of additional collagen cross-links by glutaraldehyde treatment significantly increased the bond strength of the Single Bond adhesive system to caries-affected dentin.

The inter and intra-molecular cross-linking of collagen are the basis for the stability, tensile strength and visco-elasticity of the collagen fibrils. Several synthetic and natural occurring agents have the ability to increase the number of collagen cross-links, but variations may exist among the type and the amount of exogenous cross-links induced. It has been shown that according to the cross-linking agents used, different types of cross-links (inter- and intra-molecular) can be formed in collagen matrices. Different exogenous cross-links formed in the collagen fibrils may results in differences in collagen degradation rates and mechanical properties. These are important factors to be considered when modifying the dentin collagen matrix for restorative procedures. Higher mechanical properties and lower collagen degradation rates are desirable characteristics for the bonded interface that will allow for long lasting restorations.

To determine if a decrease in the rates of collagen degradation at the restoration/dentin interface occurs when dentin is treated with cross-linking agents, regardless of the type of dentin (sound and caries-affected dentin), the experiment described below can be performed.

Specimen preparation: Sound and carious human molars (selected as described above) are collected after extraction from patients 15 to 29 years old. The teeth are cleaned and stored frozen until used. A minimum of 5 teeth per group are used. The teeth are thawed, cleaned of debris, and the occlusal surfaces are ground flat with 320 and 600 grit Silicon Carbide paper under running water to remove enamel and to expose middle dentin, respectively. For the caries-affected group, caries detector solution (Kuraray Dental Co.) are applied to the surface in order to identify sound (no staining) and caries-affected dentin (slightly stained pink), using 600 grit abrasive paper until caries-affected tissue dentin is exposed (as described above). Then, the flat dentin are acid etched using 37% phosphoric acid (3M ESPE) and the teeth are randomly divided according to the dentin treatment as described above: proanthocyanidin in PBS, genipin in PBS, glutaraldehyde in PBS, carbodiimide+NHS in PBS and only PBS (as a control).

Specimens are exposed to the different treatment for 4 hours. After the dentin treatment, specimens are subdivided according to the adhesive system used: ethanol based adhesive system (Single Bond—3M ESPE) and an acetone based adhesive system (One Step—Bisco). Two adhesive systems with different solvents and formulations are selected since they may interact differently with the dentin matrix. Then a thin layer of a low-viscosity flowable composite (Protect Liner F—Kuraray Inc., New York, N.Y.) is applied over the bonded interface to finalize the bonding procedure. Protect Liner F is chosen due to its low concentration of filler particles and therefore will avoid damage to the diamond knife used during TEM sample preparation.

Afterwards, the restorations are sectioned perpendicular to the bonded interface into 0.7±0.2 mm thick slabs. The slabs are further trimmed at the interface (sound or caries-affected dentin) by a fine diamond bur to produce a cross-sectional surface area of 1 mm². The slabs are subdivided according to the aging procedures specimens described above.

Immunogold analysis: After aging procedures elapsed, the specimens are rinsed in saline solution for 15 min, then fixed in Karnovsky's solution and post-fixed in osmium tetroxide. The specimens then are processed for embedding in epoxy resin after dehydration in ascending ethanol (30 to 100%). Finally, they are polymerized at 45° C. for 3 days in gelatin capsules. The resin blocks are trimmed and sections are obtained using an ultramicrotome apparatus (Reichert SuperNova ultramicrotome, Leica Inc, Wein, Austria) equipped with a diamond knife (MicroStar Technologies Inc. Huntsville, Tex., USA). Ultra thin sections (80 nm thick) are obtained and mounted on nickel grids. The grids are treated with 5% Na meta periodate water solution for 30 min, rinsed with water, submitted to 0.1M of HCl solution for 10 min, and rinsed in 0.05M of TBS-Tris Buffered Saline at pH 7.6. Specimens are pre-incubated for 30 min at room temperature with normal goat serum, and overnight incubation is performed using a primary monoclonal antibody (mouse anti-type I collagen, Clone Col-1, Sigma Chemical Co., St. Louis, Mo.). A secondary antibody, a goat anti-mouse IgG conjugated with 15 nm of colloidal gold particles (British BioCell International, Cardiff; UK) is applied in 0.02M of TBS at pH 8.2. The grids then are stained in 2% uranyl acetate and 3% lead citrate and the colloidal gold particles are localized and quantified using a TEM (JEOL JEM-2010F field emission, Peabody, Mass. USA). Control specimens are processed as described and then incubated with unlabeled antibodies or overnight in 0.05M of TBS at pH 7.6 without the primary antibody. A total of 15 (3 slices per specimen) analyses per group (40 groups that include the variables: two dentin substrate, five dentin treatment, one application times and four methods of aging time) are performed. Assessment of the amount of collagen fibrils is made by the quantification of the number of colloidal gold particles per 1 μm² of the sections.

Data and Statistical analysis: The amount of collagen, measured by the quantification of gold particles, is analyzed by a parametric analysis of variance—ANOVA and Fisher's PLSD test (p<0.05)

### Example 3

### Changes in Stiffness of Demineralized Dentin Following Application of Collagen Cross-Linkers

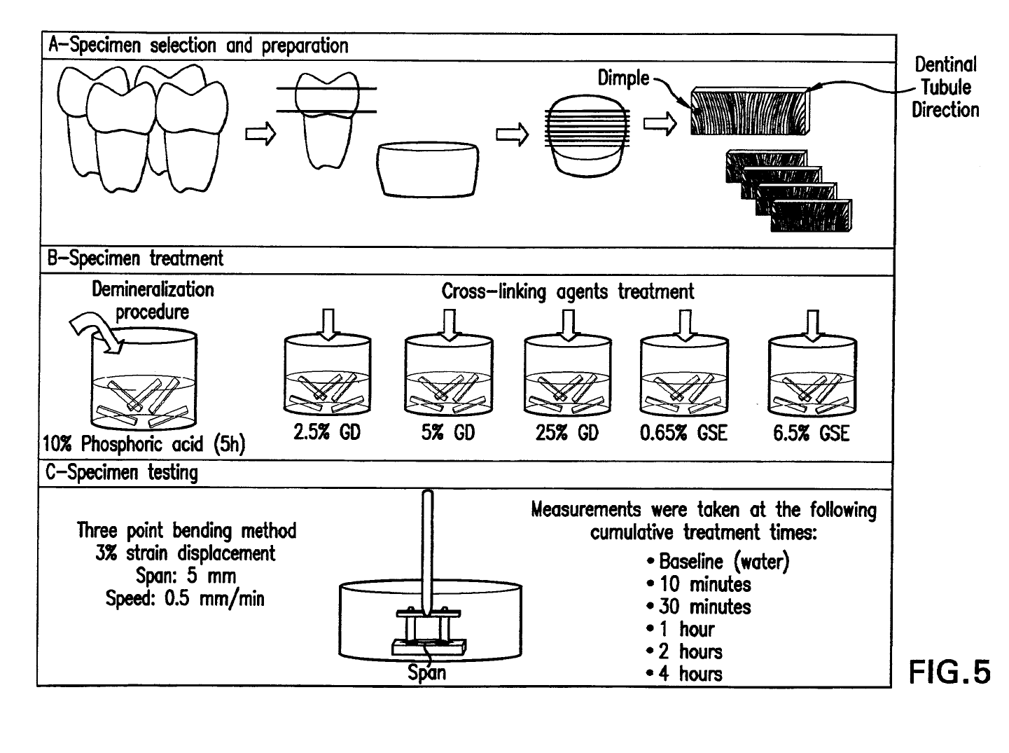

To evaluate the effect of two collagen cross-linking agents used at different concentrations and exposure times on the modulus of elasticity of demineralized coronal dentin, the experiment below was performed. FIG. 5 shows the steps of specimen selection, preparation, treatment, and testing.

Specimen Preparation

Twelve sound extracted human molars were used. The teeth were thawed, cleaned of adhering soft tissues, and the occlusal surfaces were ground flat with #320 grit silicon carbide abrasive paper under running water to remove part of the enamel (Buehler, Lake Bluff, Ill.). Flattening of the occlusal surface by cusp removal enables more accurate sectioning of samples. The root portion was sectioned 1 mm below the CEJ and discarded. Teeth were sectioned into 0.5±0.1 mm thick beams (n=5 beams per tooth) in the mesio-buccal direction with a slow speed diamond wafering blade (Buehler-Series 15LC Diamond) under constant water irrigation (FIG. 1A). The sections were further trimmed using a cylindrical diamond bur (#557D, Brasseler, Savannah, Ga.) in a high speed handpiece, to a final rectangular dimension of 0.5 mm thickness×1.7 mm width×7.0 mm length. A dimple was made at one end of the surfaces to allow for repeated measurements to be performed on the same surface (FIG. 1A).

Specimens were immersed in 10% phosphoric acid solution (LabChem Inc, Pittsburgh, Pa.) for a period of 5 hours and thoroughly rinsed with distilled water for 10 minutes. X-rays were taken of the specimens to verify complete tissue demineralization. Demineralized specimens were randomly divided into the following groups according to dentin treatment (FIG. 1B): 2.5% glutaraldehyde (Fisher Biotech, Fair Lawn, N.J.) in PBS (pH: 7.4); 5% glutaraldehyde in PBS (pH: 7.4); 25% glutaraldehyde in PBS (pH: 7.4); 0.65% grape seed extract (Mega-Natural—Polyphenolics Ind., Madera, Calif.) in PBS (pH: 7.4); and 6.5% grape seed extract in PBS solution (pH: 7.4). Specimens were immersed in water for baseline measurements and then in their respective solutions for 10 min, 30 min, 1 hour, 2 hour and 4 hours of cumulative exposure (FIG. 1C).

Modulus of Elasticity—3 Point Bend Method

The 3-point bend method was selected for evaluation of the modulus of elasticity. The advantages of this method are that it requires no gripping of these small specimens and that it is non-destructive and therefore permits repeated measurements to be performed (FIG. 1C). An aluminum alloy fixture with a 5.0 mm span between supports was glued to the bottom of a glass Petri dish. Specimens were tested in compression while immersed in liquid using a Vitrodyne testing machine (Model V-1000, Chatillon, Greensboro, N.C.) with a 50 grams load cell at crosshead speed of 0.5 mm/min. Load-displacement curves were converted to stress-strain curves and the apparent modulus of elasticity calculated at 3% strain. Displacement (D) during compression was calculated at a maximum strain of 3% using the following formula (Nielsen L E. Mechanical properties of polymers and composites. Vol. 1, New York: Marcel Dekker Inc., 1974, p. 45.):

D (mm)=εL²/6T (1)

Where ε is strain, L is support span and T is thickness of the specimen.

Then the modulus of elasticity (É) of the specimens was calculated using the following formula (Nielsen, 1974):

É(MPa)=PL³/4DbT (2)

Where P is the maximum load, L is the support span, D is the displacement, b width of the specimen and T is the thickness of the specimen.

Baseline measurements were obtained with specimens immersed in distilled water. All experimental periods were evaluated with the samples immersed in their respective cross-linking agents. A total of 10-12 specimens were evaluated per group. The data were collected and statistically analyzed using a General Linear Model SPSS program for ANOVA repeated measurements at a 95% confidence interval.

Results

The modulus of elasticity (E) values and standard deviations (MPa±SD) are shown in Table 3.

TABLE 3
Changes on the modulus of elasticity [MPa (Standard deviation)] of
human demineralized dentin matrix overtime in different concentrations
of Glutaraldehyde and grape seed extract.

| | Treatment | | | | | |
|---|---|---|---|---|---|---|
| | | | 25% | 0.65% | | Pooled |
| Time | 2.5% GD | 5% GD | GD | GSE | 6.5% GSE | data |
| Baseline | 4.82 | 5.97 | 5.32 | 6.16 | 6.14 | 5.68 a |
| | (1.97) | (3.17) | (1.41) | (2.25) | (3.79) | (2.52) |
| 10 min | 20.45 | 18.37 | 32.26 | 20.27 | 50.73 | 28.42 b |
| | (7.75) | (11.41) | (14.84) | (8.97) | (20.38) | (12.67) |
| 30 min | 30.22 | 27.25 | 37.83 | 39.10 | 98.19 | 46.52 c |
| | (10.43) | (17.51) | (15.14) | (16.52) | (39.15) | (19.75) |
| 1 h | 32.10 | 29.77 | 41.31 | 60.63 | 134.80 | 59.72 d |
| | (11.90) | (20.26) | (16.13) | (25.01) | (55.87) | (25.83) |
| 2 h | 34.44 | 33.08 | 41.21 | 88.24 | 183.42 | 76.08 e |
| | (11.75) | (19.08) | (14.86) | (32.77) | (80.69) | (31.83) |
| 4 h | 34.86 | 38.14 | 42.80 | 133.41 | 242.49 | 98.34 f |
| | (11.10) | (24.11) | (16.34) | (63.54) | (107.71) | (44.56) |
| Pooled | 26.15 A | 25.43 A | 33.45 B | 57.97 C | 119.30 D | |
| data | (9.15) | (15.92) | (14.06) | (39.22) | (51.27) | |

Same lower case letters indicate no statistical significant difference between rows (p > 0.05).
Same higher case letters indicate no statistical significant difference between columns (p > 0.05).
GSE—Grape seed extract;
GD—Glutaraldehyde

The different treatments (p=<0.0001) and exposure time (p=<0.0001) resulted in statistically significant differences among groups. A statistically significant (p<0.0001) interaction was observed between the factors studied (treatment vs. time). Mean baselines E values varied between 4.82-6.16 MPa in water; after 4 hours of treatment the values increased between 34.86-242.49, that were treatment agent dependent. It was observed that 25% glutaraldehyde treatment resulted in a significantly more rapid increase in E after 10 minutes exposure, compared to 2.5% and 5% glutaraldehyde (Table 3). After 1 hour exposure of cumulative time, a plateau in matrix stiffness was reached for all of the different concentrations of glutaraldehyde. In the present study, the use of 0.65% and 6.5% grape seed extract resulted in a statistically significant increase in the E of demineralized dentin following each time tested. The 6.5% grape seed extract resulted in the highest mean stiffness values when compared to all the other groups tested, regardless of the exposure time. The use of 6.5% grape seed extract for 10 minutes resulted in mean E values that were significantly higher (p<0.05) than any glutaraldehyde treated group tested even after 4 hours.

Different concentrations of glutaraldehyde and exposure time were evaluated in the present study to assess dose-dependence of the treatments. It was found that 25% glutaraldehyde treatment resulted in a more rapid increase in the elastic modulus of dentin matrix after 10 minutes exposure when compared to 2.5% and 5% glutaraldehyde. After 1 hour exposure time a plateau in stiffness was reached for all the different concentrations, with minor further increases in the mean values. glutaraldehyde reacts primarily with the ε-amino groups of Lys and Hyl residues within biological tissue and a network of exogenous cross-links can be induced intra and inter molecularly within collagen. Therefore, according to the data, the newly formed exogenous cross-links were effective in increasing the elastic modulus of the demineralized dentin, but the reaction appeared to be limited to the availability of free amino groups of Lys or Hyl residues, and therefore the elastic modulus values reached a plateau. The use of a high concentration of glutaraldehyde was beneficial during the 10 and 30 minute exposure time, but at 4 hours all the concentrations reached similar values.

In this Example, the use of grape seed extract resulted in a rapid and continuous increase in the modulus of elasticity of demineralized dentin following each time tested. Increasing concentrations of grape seed extract significantly increased the modulus of elasticity values, where the higher concentration of grape seed extract resulted in the highest mean stiffness values. The use of 6.5% grape seed extract for 10 minutes resulted in values higher than any glutaraldehyde treated group tested after 4 hours. The modulus of elasticity increased as exposure time increased for both concentrations not reaching a plateau. The mechanism of interaction of proanthocyanidin with collagen is different than that of glutaraldehyde. Four mechanisms for interaction between proanthocyanidin and proteins have been proposed including covalent interactions, ionic interactions, hydrogen bonding interactions or hydrophobic interactions. The increase in stiffness of dentin matrix between approximately 8 to 40 times following treatment with 6.5% grape seed extract for 10 minutes and 4 hours, respectively, indicates an ability of the agent to stiffen the demineralized dentin matrix.

Conclusion: Demineralized dentin stiffness is affected by the use of glutaraldehyde and grape seed extract collagen cross-linking agents. The changes to the dentin matrix after treatment with the cross-linkers were both concentration and time dependent.

### Example 4

### Effect of Collagen Cross-Linkers and Tubule Orientation on Dentin Strength

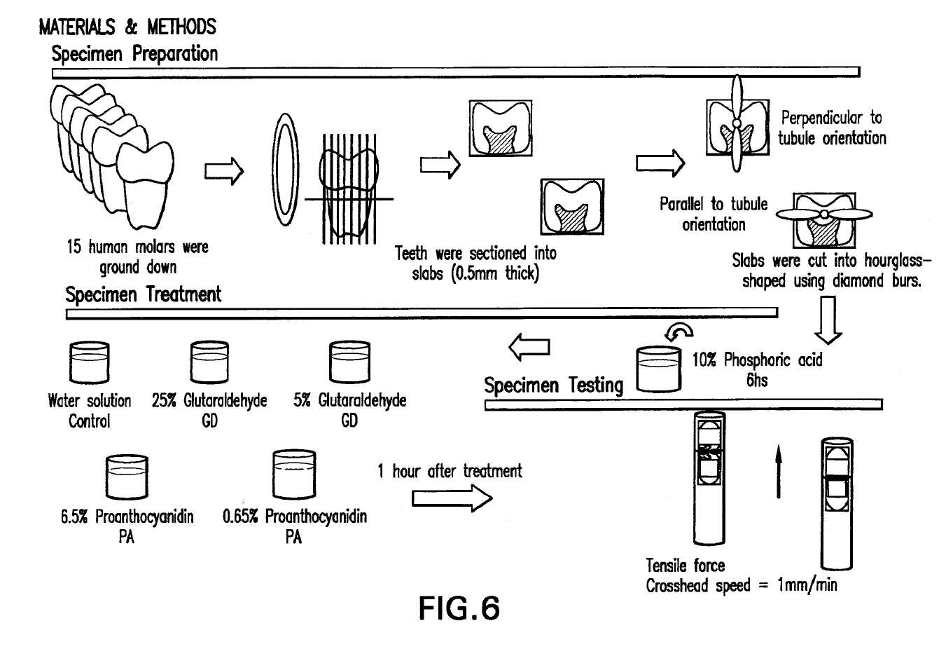

Specific synthetic and natural chemicals may increase the number of collagen cross-links within tooth dentin. To evaluate the effects of two cross-linking agents of different concentrations on the UTS of demineralized crown dentin tested parallel and perpendicular to dentinal tubules, the experiment described below was performed. FIG. 6 illustrates the steps of specimen preparation and treatment.

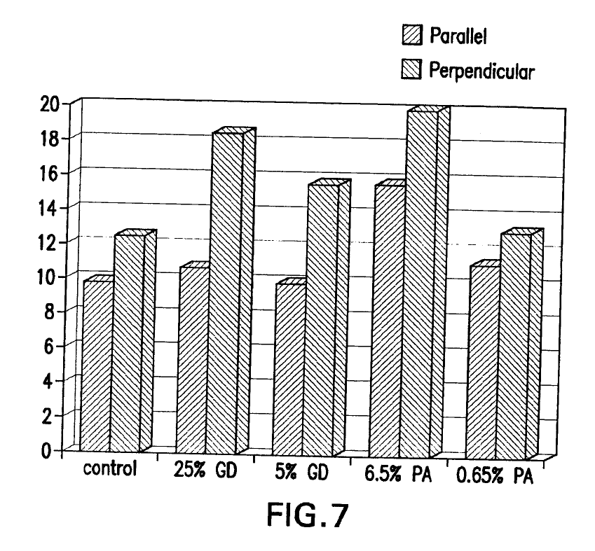

Twenty sound molars were sectioned into 0.5±0.1 mm thick slabs that were further trimmed to 0.5±0.1 mm according to the tubule orientation (parallel-PR or perpendicular-PE). Specimens were demineralized with 10% phosphoric acid and divided into 10 groups according to treatment: control (water solution)/PR; control/PE, 6.5% proanthocyanidin (PA)/PR, 6.5% PA/PE; 0.65% PA/PR; 0.65% PA/PE; 25% glutaraldehyde (GD)/PR; 25% GD/PE; 5% GD/PR; 5% GD/PE. Specimens were kept in their respective solutions for 1 hour. To assess UTS, specimens were subjected to tensile forces at a crosshead speed of 1 mm/min. Statistical analysis was performed using two-way ANOVA and Fisher's PLSD test (p<0.05). Results: UTS values [MPa (SD)] are as follows in Table 4 below and shown in FIG. 7.

TABLE 4
UTS Values

| | Treatment | | | | |
|---|---|---|---|---|---|
| Orientation | Control | 25% GD | 5% GD | 6.5% PA | 0.65% PA |
| Parallel | 9.8 ± 2.8^{D,a} | 10.7 ± 2.7^{B,b} | 9.8 ± 1.5^{BCD,b} | 15.7 ± 3.6^{A,b} | 11.1 ± 2.6^{CD,b} |
| Perpendicular | 12.44 ± 2.4^{D,a} | 18.6 ± 5.7^{B,a} | 15.7 ± 4.9^{BCD,a} | 20.0 ± 4.9^{A,a} | 13.1 ± 3.2^{CD,b} |

*Different upper and lower case letters indicate statistically significant differences on rows and columns respectively.

A statistically significant interaction was observed between the factors (p=0.0125). Orientation of dentinal tubules and treatment significantly affected the UTS (p<0.0001). Samples oriented PE to the dentinal tubules presented higher statistically significant UTS values when compared to PR, regardless of the treatment. All treatments significantly affected the UTS values of samples oriented PE, except for 0.65% PA. Treatment with 6.5% PA resulted in increased UTS values for PR direction. The application of collagen cross-linkers (PA and GD) affected the UTS of demineralized dentin, which was dependent upon tubule orientation, type and concentration of the agent.

### Example 5

### Stability of Dentin Bonding after Application of a Collagen Cross-Linker

To evaluate the effect of glutaraldehyde on the tensile bond strength (TBS) of a total-etch adhesive with or without proteolytic challenge, the experiment described below was performed.

Ten human molars were polished until occlusal dentin was exposed and were divided according to the surface treatment: a control (no treatment) treatment (G1) and a 5% glutaraldehyde treatment (G2). Samples of G1 were etched with 37% phosphoric acid, rinsed, blotted dried, and One-Step (Bisco, Richmond, BC, Canada) was applied, followed by application of a composite resin used to build-up a “crown.” G2 was treated in the same manner, except that after etching, 5% glutaraldehyde was applied on the surface for 1 hour. After 24 hours, samples were sectioned into 1.0×1.0 mm beams. The beams were subdivided according to the proteolytic challenge: stored in water for 2 hours (no challenge) and stored in 1.5% NaOCl solution for 2 hours. Then samples were rinsed and tested in tensile at a crosshead speed of 1 mm/min using a universal testing machine. Data were statistically analyzed using ANOVA and Fisher's PLSD tests (p<0.05). Results: TBS values [MPa (SD)] are as follows in Table 5 below:

TABLE 5
TBS Values

Proteolytic challenge

| Treatment | No challenge | 1.5% NaOCl |
|---|---|---|
| Control | 63.45 (7.91)B | 42.01 (11.82)C |
| 5% Glutaraldehyde | 71.11 (12.20)A | 65.91 (18.06)AB |

A statistically significant interaction was observed between the factors (p=0.0215). Surface treatment and proteolytic challenge significantly affected the TBS (p<0.0001 and p=0.003, respectively). Treatment of the surface with 5% glutaraldehyde resulted in increased TBS when compared to the control group. After NaOCl storage, there was a significant decrease in the TBS values for the control, while 5% glutaraldehyde was not significantly affected. The use of 5% glutaraldehyde resulted in increased TBS and stability of the bond after proteolytic challenge.

### Example 6

### Remineralization Effects of CPP-ACP and Proanthocyanidin on Artificial Root Caries

Root caries is of major concern in dentistry since it is the most common oral disease in the elderly. To evaluate the in vitro effect of CPP-ACP-based paste (MI-GC) and proanthocyanidin (MegaNatural-Polyphenolics, Madera, Calif.) on the remineralization of root dentin, the below-described hardness test was performed.

Sound human teeth fragments obtained from the cervical portion of the root were stored in demineralization solution (pH 4.6) for 96 hours at 37° C. to produce artificial dentin carious lesions. Then, the samples were pH-cycled through treatment solutions. An acidic buffer (pH 5.0) and a neutral buffer (pH 7.0) were used during 8 days. The fragments were divided according to the treatment: control (no treatment); 6.5% proanthocyanidin; MI; and 6.5% proanthocyanidin+MI. Six cycles per day were done as follows: 30 minutes acidic solution, 10 minutes neutral solution, and 10-20 minutes treatment with respective agents. Afterwards, samples were sectioned, embedded in epoxy resin and polished. Cross-sectional microhardness measurements were performed at the subsurface using Knoop indentation (KHN) at 25 grams load force for 15 seconds. Data was analyzed using ANOVA and Fisher's tests (p<0.05).

Result: The microhardness values were as follow [mean (SD)]:

TABLE 6
Microhardness Values

| | Depth (KHN) | | |
|---|---|---|---|
| Treatment | 10 um | 30 um | Pooled data |
| control | 32.12 (9.86) | 52.39 (12.80) | 42.25 (11.33)B |
| MI | 31.39 (11.05) | 49.50 (10.48) | 40.45 (10.76)B |
| PA | 36.38 (9.48) | 46.81 (8.10) | 41.60 (8.79)B |
| PA + MI | 38.83 (12.13) | 56.57 (10.58) | 47.70 (11.35)A |
| Pooled data | 34.68 (10.63) B | 51.32 (10.49)A | |

*Different letters indicate statistically significant differences

There was no statistically significant interaction among the factors studied: treatment vs. depth (p=0.192). Depth and treatment significantly affected the hardness values (p<0.0001 and p=0.0222, respectively). The combination of a CPP-ACP-based paste and proanthocyanidin resulted in increased hardness values. CPP-ACP-based paste and proanthocyanidin can be used during non-invasive remineralization therapy.

### Example 7

### Effect of Grape Seed Extract and Glutaraldehyde on the Tensile Strength of Demineralized Dentin after Collagenase Challenge

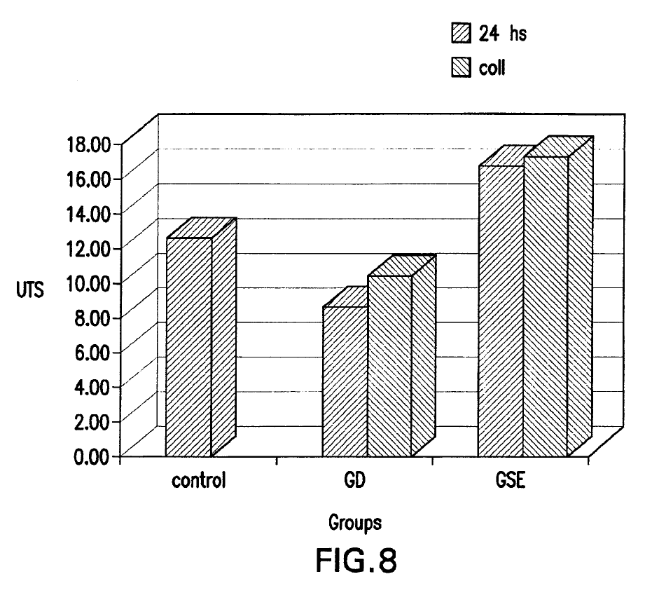

The data shown below in Tables 7 to 14 and in FIG. 8 describes the effect of grape seed extract and Glutaraldehyde on the tensile strength of demineralized dentin after collagenase challenge. Samples were treated and then subjected to collagenase digestion. The control groups were fully digested while the grape seed extract and Glutaraldehyde groups were not affected by the collagenase treatment and the strength values remained similar to baseline (no collagenase treatment). The data shows that grape seed extract and Glutaraldehyde protected the demineralized dentin against enzymatic degradation. Grape seed extract specifically increased the tensile values and protected the dentin against degradation.

TABLE 7
Study: Evaluate the effect of 6.5% GSE and 25% GD on the UTS of demineralized dentin after aging

| Group 1 | treatment | challenge | specimen | Thick (mm) | width (mm) | Area (mm2) | Peak Break | Mpa | Fracture |
|---|---|---|---|---|---|---|---|---|---|
| control | 1 h DW | 24 hrs | 1 | 0.32 | 0.95 | 0.30 | 0.35 | 11.28 | |
| control | 1 h DW | 24 hrs | 2 | 0.45 | 0.84 | 0.38 | 0.34 | 8.81 | |
| control | 1 h DW | 24 hrs | 3 | 0.42 | 1.00 | 0.42 | 0.86 | 20.07 | near glue |
| control | 1 h DW | 24 hrs | 4 | 0.46 | 0.80 | 0.37 | 0.39 | 10.39 | |
| control | 1 h DW | 24 hrs | 5 | 0.43 | 1.00 | 0.43 | 0.51 | 11.62 | near glue |
| control | 1 h DW | 24 hrs | 6 | 0.39 | 1.03 | 0.40 | 0.59 | 14.39 | |
| control | 1 h DW | 24 hrs | 7 | 0.46 | 0.96 | 0.44 | 0.30 | 6.66 | |
| control | 1 h DW | 24 hrs | 8 | 0.42 | 0.99 | 0.42 | 0.66 | 15.56 | |
| control | 1 h DW | 24 hrs | 9 | 0.44 | 1.00 | 0.44 | 0.95 | 21.16 | |
| control | 1 h DW | 24 hrs | 10 | 0.42 | 0.99 | 0.42 | 0.80 | 18.86 | near glue |
| control | 1 h DW | 24 hrs | 11 | 0.41 | 0.85 | 0.35 | 0.27 | 7.59 | |
| control | 1 h DW | 24 hrs | 12 | 0.44 | 0.86 | 0.38 | 0.38 | 9.84 | |
| control | 1 h DW | 24 hrs | 13 | 0.47 | 1.01 | 0.47 | 0.72 | 14.86 | |
| control | 1 h DW | 24 hrs | 14 | 0.49 | 0.54 | 0.26 | 0.17 | 6.30 | |
| | | | | | mean (area) | 0.39 | mean (peak) | 12.67 | |
| | | | | | SD | 0.06 | SD | 4.92 | |

TABLE 8
Study: Evaluate the effect of 6.5% GSE and 25% GD on the UTS of demineralized dentin after aging

| Group 2 | treatment | challenge | specimen | Thick (mm) | width (mm) | Area (mm2) | Peak Break | Mpa | Fracture |
|---|---|---|---|---|---|---|---|---|---|
| GD | 1 h 25% DW | 24 hrs | 1 | 0.39 | 1.05 | 0.41 | 0.50 | 11.97 | near glue |
| GD | 1 h 25% DW | 24 hrs | 2 | 0.41 | 0.91 | 0.37 | 0.34 | 8.93 | |
| GD | 1 h 25% DW | 24 hrs | 3 | 0.48 | 0.86 | 0.41 | 0.32 | 7.60 | |
| GD | | | | | | | | | |
| GD | 1 h 25% DW | 24 hrs | 5 | 0.44 | 1.01 | 0.44 | 0.38 | 8.38 | |
| GD | 1 h 25% DW | 24 hrs | 6 | 0.51 | 1.01 | 0.52 | 0.89 | 16.93 | near glue |
| GD | 1 h 25% DW | 24 hrs | 7 | 0.51 | 0.91 | 0.46 | 0.25 | 5.28 | |
| GD | 1 h 25% DW | 24 hrs | 8 | 0.37 | 0.94 | 0.35 | 0.35 | 9.86 | near glue |
| GD | 1 h 25% DW | 24 hrs | 9 | 0.46 | 0.98 | 0.45 | 0.44 | 9.57 | |
| GD | 1 h 25% DW | 24 hrs | 10 | 0.40 | 0.81 | 0.32 | 0.25 | 7.56 | |
| GD | 1 h 25% DW | 24 hrs | 11 | 0.42 | 1.02 | 0.43 | 0.36 | 8.24 | |
| GD | 1 h 25% DW | 24 hrs | 12 | 0.45 | 0.97 | 0.44 | 0.39 | 8.76 | |
| GD | 1 h 25% DW | 24 hrs | 13 | 0.49 | 0.99 | 0.49 | 0.28 | 5.66 | |
| GD | 1 h 25% DW | 24 hrs | 14 | 0.40 | 0.92 | 0.37 | 0.30 | 7.99 | |
| | | | | | mean (area) | 0.42 | mean (peak) | 8.98 | |
| | | | | | SD | 0.06 | SD | 2.94 | |

TABLE 9
Study: Evaluate the effect of 6.5% GSE and 25% GD on the UTS of demineralized dentin after aging

| Group 3 | treatment | challenge | specimen | Thick (mm) | width (mm) | Area (mm2) | Peak Break | Mpa | Fracture |
|---|---|---|---|---|---|---|---|---|---|
| GSE | 1 h 6.5% DW | 24 hrs | 1 | 0.46 | 1.00 | 0.46 | 0.58 | 12.36 | |
| GSE | 1 h 6.5% DW | 24 hrs | 2 | 0.36 | 0.87 | 0.31 | 0.56 | 17.52 | near glue |
| GSE | 1 h 6.5% DW | 24 hrs | 3 | 0.42 | 0.99 | 0.42 | 0.77 | 18.15 | |
| GSE | 1 h 6.5% DW | 24 hrs | 4 | 0.45 | 0.78 | 0.35 | 0.40 | 11.17 | |
| GSE | 1 h 6.5% DW | 24 hrs | 5 | 0.45 | 1.01 | 0.45 | 0.78 | 16.82 | |
| GSE | 1 h 6.5% DW | 24 hrs | 6 | 0.47 | 1.02 | 0.48 | 0.59 | 12.06 | |
| GSE | 1 h 6.5% DW | 24 hrs | 7 | 0.47 | 0.91 | 0.43 | 0.69 | 15.81 | |
| GSE | 1 h 6.5% DW | 24 hrs | 8 | 0.52 | 1.03 | 0.54 | 0.80 | 14.64 | |
| GSE | 1 h 6.5% DW | 24 hrs | 9 | 0.45 | 0.95 | 0.43 | 0.58 | 13.30 | |
| GSE | 1 h 6.5% DW | 24 hrs | 10 | 0.42 | 0.91 | 0.38 | 0.84 | 21.54 | |
| GSE | 1 h 6.5% DW | 24 hrs | 11 | 0.47 | 0.98 | 0.46 | 0.89 | 18.94 | |
| GSE | 1 h 6.5% DW | 24 hrs | 12 | 0.42 | 0.99 | 0.42 | 0.69 | 16.26 | |
| GSE | 1 h 6.5% DW | 24 hrs | 13 | 0.51 | 0.89 | 0.45 | 0.80 | 17.27 | |
| GSE | 1 h 6.5% DW | 24 hrs | 14 | 0.52 | 0.81 | 0.42 | 0.74 | 17.22 | |
| GSE | 1 h 6.5% DW | 24 hrs | 15 | 0.40 | 0.50 | 0.20 | 0.48 | 23.52 | ** |
| GSE | 1 h 6.5% DW | 24 hrs | 16 | 0.44 | 0.51 | 0.22 | 0.50 | 21.84 | ** |
| | | | | | mean (area) | 0.40 | mean (peak) | 16.78 | |
| | | | | | SD | 0.09 | SD | 3.58 | |

TABLE 10
Study: Evaluate the effect of 6.5% GSE and 25% GD on the UTS of demineralized dentin after aging
Apr. 25, 2007

| Group 4 | treatment | challenge | specimen | Thick (mm) | width (mm) | Area (mm2) | Peak Break | Mpa | Fracture |
|---|---|---|---|---|---|---|---|---|---|
| control | 1 h DW | 24 hrs coll | 1 | 0.46 | 0.77 | 0.35 | 0.00 | 0.00 | |
| control | 1 h DW | 24 hrs coll | 2 | 0.47 | 1.01 | 0.47 | 0.00 | 0.00 | |
| control | 1 h DW | 24 hrs coll | 3 | 0.43 | 0.94 | 0.40 | 0.00 | 0.00 | |
| control | 1 h DW | 24 hrs coll | 4 | 0.44 | 0.99 | 0.44 | 0.00 | 0.00 | |
| control | 1 h DW | 24 hrs coll | 5 | 0.46 | 0.90 | 0.41 | 0.00 | 0.00 | |
| control | 1 h DW | 24 hrs coll | 6 | 0.30 | 0.98 | 0.29 | 0.00 | 0.00 | |
| control | 1 h DW | 24 hrs coll | 7 | 0.43 | 0.84 | 0.36 | 0.00 | 0.00 | |
| control | 1 h DW | 24 hrs coll | 8 | 0.46 | 0.95 | 0.44 | 0.00 | 0.00 | |
| control | 1 h DW | 24 hrs coll | 9 | 0.45 | 0.98 | 0.44 | 0.00 | 0.00 | |
| control | 1 h DW | 24 hrs coll | 10 | 0.47 | 1.01 | 0.47 | 0.00 | 0.00 | |
| control | 1 h DW | 24 hrs coll | 11 | 0.43 | 0.89 | 0.38 | 0.00 | 0.00 | |
| control | 1 h DW | 24 hrs coll | 12 | 0.95 | 0.95 | 0.90 | 0.00 | 0.00 | |
| control | 1 h DW | 24 hrs coll | 13 | 0.45 | 0.94 | 0.42 | 0.00 | 0.00 | |
| | | | | | mean (area) | 0.45 | mean (peak) | 0.00 | |
| | | | | | SD | 0.15 | SD | 0.00 | |

TABLE 11
Study: Evaluate the effect of 6.5% GSE and 25% GD on the UTS of demineralized dentin after aging

| Group 5 | treatment | challenge | specimen | Thick (mm) | width (mm) | Area (mm2) | Peak Break | Mpa | Fracture |
|---|---|---|---|---|---|---|---|---|---|
| GD | 1 h 25% DW | 24 hrs coll | 1 | 0.46 | 1.01 | 0.46 | 0.82 | 17.30 | near glue |
| GD | 1 h 25% DW | 24 hrs coll | 2 | 0.48 | 0.87 | 0.42 | 0.33 | 7.74 | |
| GD | 1 h 25% DW | 24 hrs coll | 3 | 0.45 | 0.96 | 0.43 | 0.43 | 9.75 | |
| GD | 1 h 25% DW | 24 hrs coll | 4 | 0.43 | 1.05 | 0.45 | 0.29 | 6.29 | |
| GD | 1 h 25% DW | 24 hrs coll | 5 | 0.48 | 0.84 | 0.40 | 0.68 | 16.53 | |
| GD | 1 h 25% DW | 24 hrs coll | 6 | 0.49 | 0.97 | 0.48 | 0.50 | 10.31 | near glue |
| GD | 1 h 25% DW | 24 hrs coll | 7 | 0.40 | 0.80 | 0.32 | 0.54 | 16.54 | |
| GD | 1 h 25% DW | 24 hrs coll | 8 | 0.35 | 0.90 | 0.32 | 0.28 | 8.71 | near glue |
| GD | 1 h 25% DW | 24 hrs coll | 9 | 0.42 | 1.02 | 0.43 | 0.37 | 8.46 | |
| GD | 1 h 25% DW | 24 hrs coll | 10 | 0.47 | 0.97 | 0.46 | 0.26 | 5.59 | |
| GD | 1 h 25% DW | 24 hrs coll | 11 | 0.51 | 1.01 | 0.52 | 0.43 | 8.18 | |
| GD | 1 h 25% DW | 24 hrs coll | 12 | 0.43 | 0.91 | 0.39 | 0.37 | 9.27 | |
| GD | 1 h 25% DW | 24 hrs coll | 13 | 0.37 | 1.01 | 0.37 | 0.47 | 12.33 | |
| | | | | | mean (area) | 0.42 | mean (peak) | 10.54 | |
| | | | | | SD | 0.06 | SD | 3.94 | |

TABLE 12
Study: Evaluate the effect of 6.5% GSE and 25% GD on the UTS of demineralized dentin after aging

| Group 6 | treatment | challenge | specimen | Thick (mm) | width (mm) | Area (mm2) | Peak Break | Mpa | Fracture |
|---|---|---|---|---|---|---|---|---|---|
| GSE | 1 h 6.5% DW | 24 hrs coll | 1 | 0.44 | 0.91 | 0.40 | 1.02 | 24.97 | |
| GSE | 1 h 6.5% DW | 24 hrs coll | 2 | 0.45 | 1.03 | 0.46 | 0.85 | 17.97 | |
| GSE | 1 h 6.5% DW | 24 hrs coll | 3 | 0.45 | 0.80 | 0.36 | 0.50 | 13.61 | |
| GSE | 1 h 6.5% DW | 24 hrs coll | 4 | 0.49 | 0.94 | 0.46 | 0.65 | 13.83 | |
| GSE | 1 h 6.5% DW | 24 hrs coll | 5 | 0.47 | 0.99 | 0.47 | 0.67 | 14.11 | |
| GSE | 1 h 6.5% DW | 24 hrs coll | 6 | 0.41 | 0.91 | 0.37 | 0.89 | 23.38 | |
| GSE | 1 h 6.5% DW | 24 hrs coll | 7 | 0.39 | 0.87 | 0.34 | 0.64 | 18.49 | |
| GSE | 1 h 6.5% DW | 24 hrs coll | 8 | 0.52 | 1.02 | 0.53 | 0.66 | 12.19 | |
| GSE | 1 h 6.5% DW | 24 hrs coll | 9 | 0.39 | 0.97 | 0.38 | 0.56 | 14.51 | |
| GSE | 1 h 6.5% DW | 24 hrs coll | 10 | 0.43 | 0.92 | 0.40 | 0.61 | 15.11 | |
| GSE | 1 h 6.5% DW | 24 hrs coll | 11 | 0.45 | 0.90 | 0.41 | 0.85 | 20.57 | |
| GSE | 1 h 6.5% DW | 24 hrs coll | 12 | 0.44 | 0.98 | 0.43 | 0.82 | 18.64 | |
| GSE | 1 h 6.5% DW | 24 hrs coll | 13 | 0.37 | 1.01 | 0.37 | 0.58 | 15.21 | |
| | | | | | mean (area) | 0.41 | mean (peak) | 17.12 | |
| | | | | | SD | 0.05 | SD | 3.96 | |

TABLE 13
Study: Evaluate the effect of 6.5% GSE and 25% GD on the UTS of
demineralized dentin after aging

| | |
|---|---|
| substrate: 12 teeth (6-8 sections demineralized sections | sections size: |
| per tooth) | approximately |
| | 0.04 mm2 |
| demineralization: 10% phosphoric acid for 6 hours | |
| Groups: | |
| 24 hours (amonium bicarbonate buffer) | |
| control (no treat) | |
| 25% Glutaraldehyde (25% GD) | |
| 6.5% Grape Seed Extract (6.5% GSE) | |
| 24 hours (collagenase + amonium bicarbonate buffer) | concentration: |
| | 100 ug/ml |
| control (no treat) | |
| 25% Glutaraldehyde (25% GD) | |
| 6.5% Grape Seed Extract (6.5% GSE) | |

TABLE 14

| | mean (Mpa) | SD (Mpa) |
|---|---|---|
| control | 12.67 | 4.92 |
| GD | 8.98 | 2.94 |
| GSE | 16.78 | 3.58 |
| control/coll | 0.00 | 0 |
| GD/coll | 10.54 | 3.94 |
| GSE/coll | 17.12 | 3.96 |

| | 24 hs | coll |
|---|---|---|
| control | 12.67 | 0 |
| GD | 8.99 | 10.54 |
| GSE | 16.78 | 17.12 |

### OTHER EMBODIMENTS

Any improvement may be made in part or all of the components. All references, including publications, patent applications, and patents, cited herein are hereby incorporated by reference. In any listing of possible components, mixtures of possible components are contemplated unless expressly indicated otherwise. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A composition for increasing collagen cross-linking in a mammalian tissue comprising at least one of: a bioflavonoid compound, a grape seed extract, a Casein Phosphopeptide-amorphous Calcium Phosphate, a synthetic collagen cross-linker and an iridoid compound in a pharmaceutically acceptable carrier,
wherein the at least one of: a bioflavonoid compound, a grape seed extract, a Casein Phosphopeptide-amorphous Calcium Phosphate, a synthetic collagen cross-linker and an iridoid compound is present in an amount effective for increasing collagen cross-linking in the mammalian tissue.

2. The composition of claim 1, wherein the at least one of: a bioflavonoid compound, a grape seed extract, a Casein Phosphopeptide-amorphous Calcium Phosphate, a synthetic collagen cross-linker and an iridoid compound is a bioflavonoid compound.

3. The composition of claim 2, wherein the bioflavonoid compound is proanthocyanidin.

4. The composition of claim 1, wherein the at least one of: a bioflavonoid compound, a grape seed extract, a Casein Phosphopeptide-amorphous Calcium Phosphate, a synthetic collagen cross-linker and an iridoid compound is a synthetic collagen cross-linker.

5. The composition of claim 4, wherein the synthetic collagen cross-linker is glutaraldehyde.

6. The composition of claim 1, wherein the composition comprises a grape seed extract and a Casein Phosphopeptide-amorphous Calcium Phosphate.

7. The composition of claim 1, wherein the at least one of: a bioflavonoid compound, a grape seed extract, a Casein Phosphopeptide-amorphous Calcium Phosphate, a synthetic collagen cross-linker and an iridoid compound is an iridoid compound.

8. The composition of claim 7, wherein the iridoid compound is selected from the group consisting of: genipin, canthoside, caudatoside, saprosmoside, bis-iridoid, aucubin, gardenoside, alpha-iridodiol, geniposide, villoside, patrinoside aglycone, 11-deoxy patrinoside aglycone, gardenogenins, deacetylasperulosidic acid methylester genins, scandosife methylester genin, rehmannoside, harpagoside, harpagide, loganin, loganic acid, cantleyoside, secologanin, and secologanin-dimethylacetal.

9. The composition of claim 1, wherein the at least one of: a bioflavonoid compound, a grape seed extract, a Casein Phosphopeptide-amorphous Calcium Phosphate, a synthetic collagen cross-linker and an iridoid compound is a grape seed extract.

10. The composition of claim 9, wherein the grape seed extract comprises proanthocyanidins.

11. The composition of claim 1, wherein the mammalian tissue is dentin.

12. The composition of claim 11, wherein the collagen is type I collagen.

13. The composition of claim 1, wherein the pharmaceutically acceptable carrier is selected from the group consisting of: distilled water, phosphate buffered solution, a solvent, a gel, and an acid.

14. The composition of claim 13, wherein the acid is selected from the group consisting of: phosphoric acid, and citric acid.

15. The composition of claim 14, wherein the solvent is alcohol.

16. The composition of claim 13, wherein the gel is carbopol.

17. The composition of claim 1, wherein the at least one of: a bioflavonoid compound, a grape seed extract, a Casein Phosphopeptide-amorphous Calcium Phosphate, a synthetic collagen cross-linker and an iridoid compound is a grape seed extract and the amount effective for increasing collagen cross-linking in the mammalian tissue is about 0.001% to about 30% weight by volume.

18. The composition of claim 1, wherein the at least one of: a bioflavonoid compound, a grape seed extract, a Casein Phosphopeptide-amorphous Calcium Phosphate, a synthetic collagen cross-linker and an iridoid compound is an iridoid compound and the amount effective for increasing collagen cross-linking in the mammalian tissue is about 0.001% to about 5% weight by volume.

19. The composition of claim 18, wherein the iridoid compound is genipin.

20. The composition of claim 1, wherein the at least one of: a bioflavonoid compound, a grape seed extract, a Casein Phosphopeptide-amorphous Calcium Phosphate, a synthetic collagen cross-linker and an iridoid compound is a synthetic cross-linker consisting of glutaraldehyde and the amount effective for increasing collagen cross-linking in the mammalian tissue is about 0.001% to about 25% weight by volume.

21. A method of increasing collagen cross-linking in dentin in a mammalian tooth comprising the steps of:
(a) preparing the surface of the tooth to be treated; and
(b) applying a composition comprising at least one of: a bioflavonoid compound, a grape seed extract, a Casein Phosphopeptide-amorphous Calcium Phosphate, and an iridoid compound in a pharmaceutically acceptable carrier to the tooth surface for a time period of 0.0001 hours to about 4 hours,
wherein the at least one of: a bioflavonoid compound, a grape seed extract, a Casein Phosphopeptide-amorphous Calcium Phosphate, a synthetic collagen cross-linker and an iridoid compound is present in an amount effective for increasing collagen cross-linking in the mammalian tissue.

22. The method of claim 21, wherein the step (a) of preparing the tooth surface includes etching the tooth surface with a reagent.

23. The method of claim 22, wherein the reagent comprises phosphoric acid.

24. The method of claim 21, wherein the at least one of: a bioflavonoid compound, a grape seed extract, a Casein Phosphopeptide-amorphous Calcium Phosphate, a synthetic collagen cross-linker and an iridoid compound is grape seed extract.

25. The method of claim 21, wherein the at least one of: a bioflavonoid compound, a grape seed extract, a Casein Phosphopeptide-amorphous Calcium Phosphate, a synthetic collagen cross-linker and an iridoid compound is an iridoid compound.

26. The method of claim 25, wherein the iridoid compound is genipin.

27. A method of performing a restorative procedure on a mammalian tooth comprising the steps of:
(a) preparing the surface of the tooth to be treated;
(b) applying a composition comprising at least one of: a bioflavonoid compound, a grape seed extract, a Casein Phosphopeptide-amorphous Calcium Phosphate, and an iridoid compound in a pharmaceutically acceptable carrier to the tooth surface for a time period of 0.0001 hours to about 4 hours,
wherein the at least one of: a bioflavonoid compound, a grape seed extract, a Casein Phosphopeptide-amorphous Calcium Phosphate, a synthetic collagen cross-linker and an iridoid compound is present in an amount effective for increasing collagen cross-linking in the tooth; and
(c) bonding a restorative material to the treated tooth surface.
